(19) United States
(12) Patent Application Publication
(10) Pub. No.: US 2008/0286766 A1
(43) Pub. Date: Nov. 20, 2008

(54) Comparing Method for Expression Amount of the Same Gene from Different Sources by Base Sequence Measurement

(76) Inventor: Guohua Zhou, Nanjing (CN)

Correspondence Address: GLOBAL IP SERVICES, 7285 W. Eagle Court, Winton, CA, 95388, US

(21) Appl. No.: 11/571,827

(22) PCT Filed: Nov. 22, 2004
(86) PCT No.: PCT/CN2004/001333

§ 371 Date: Jan. 8, 2007

(30) Foreign Application Priority Data
Jul. 9, 2004 (CN) 200410062751.2

(51) Int. Cl. C12Q 1/68

(52) U.S. Cl. 435/6

(57) ABSTRACT

The present invention relates to an analysis method used to quantitatively compare the expression levels of the same gene from different sources. The method of the present invention can be used to quantitatively compare the gene expression level difference of the same gene of tissues or cells from different sources, making use of the quantitative characteristics of bioluminescent assay and the principle of adding different deoxyribonucleic acids (dNTP) one by one. The concrete steps are: reverse transcript the messenger ribonucleic acids (mRNA) from different sources into cDNA, and label a segment of source specific sequence in cDNA from each source; mix the labeled cDNA of different sources into one tube and use it as the substrate of polymerase chain reaction (PCR); PCR amplification is performed using the same common primer and a gene-specific primer; Detect the base sequence by bioluminescent assay, wherein the base type represents the different gene source, and the signal intensity of each base represents the gene expression level from each source. This method has a significant meaning for the screening of disease-related genes, clinical early diagnosis and the preparation of specific medicine for the treatment of disease.

### CROSS REFERENCE TO THE RELATED PATENT APPLICATION

This application claims the priority of the Chinese application No. 200410062751.2, filed on Jul. 9, 2004.

### FIELD OF THE INVENTION

The present invention relates to a method used to quantitatively compare the relative expression level of the same gene of tissues or cells from different sources. Specifically, it is a base sequencing method for the determination of the relative content of each DNA fragment in the mixture of DNA fragments labeled by different base sequences and all the determinations can be carried out at the same time.

### BACKGROUND OF THE INVENTION

With the progress in molecular biology and analytical apparatus, the sequencing work of human genome project (HGP) has already been finished. As the structure of the whole human genome is clarified, the next step is to analyze gene functions coded in genomes^{[1]}, gene function analysis includes the understanding the distribution of the gene transcription products mRNA and the quantity, distribution and function of proteins (the translated products of mRNA) in a cell or different organs in a body. We can look for and find the disease-related genes by comparing the gene expression levels between healthy persons and patients, and further make them used in clinical early diagnosis^{[2]}. In drug screening process, the target of drug can be found by detecting the relative gene levels of the administration group and the untreated group, and further look for and prepare the specific medicine for the treatment of disease^{[3]}. Therefore, the differential analysis of gene expression level has become one of the main tasks of the “post-sequencing age”. The developed countries have invested a lot of material resources and money to rank top in this field and further monopoly the technology. At present the major analysis methods for the gene expression level comparison are: SAGE method^{[4]}, RT-PCR (reverse transcription-polymerase chain reaction) method^{[5]} and microarray (gene chip)^{[6]}, etc. But these methods still have some drawbacks: only the gene expression levels of two individuals can be compared at a time; the prices of apparatus are very high; the operation is complex and the quantitative characteristics are bad, etc. For example, in the case of SAGE method, detection is very tedious and there are too many steps so it is hard to control, in addition, the cost is also very high, all these lead to its small popularity. RT-PCR method needs special apparatus and internal standard, its detection is also tedious and the repeatability is bad. Microarray is a high-throughput detection method, although the detection amount is large and multiple genes can be detected on one chip at the same time, samples should be labeled by fluorescent dyes, the sensitivity is bad, apparatus are expensive and the data processing is complex, therefore, it is hard to accurately compare the gene expression levels of a given gene from different sources.

It is a new developed method to determine base sequence by bioluminescence technology^{[7-8]}. This method is convenient and rapid, and has the advantages of cheap apparatus, low cost and easy to realize automation. But this method is limited to analyze the mutation and polymorphism of genes for it can only detect 10 to 30 base sequences^{[9]}.

### SUMMARY OF THE INVENTION

The purpose of the present invention is to study a method for the assay of relative gene expression level of a given gene from different sources by base sequencing technology. That's to say, how to detect the relative gene expression levels by detecting several base sequences, and establish a convenient method with high sensitivity, accurate quantification and low cost, which can be used in clinical diagnosis.

Technological solutions of the invention are as below, and the detection principle is shown in FIG. 1:

(1) Label a Given Gene from Different Sources by Base Sequencing method.
This can be realized by two methods. The first method is DNA adapter labeling method. That's to say, first extract the total RNA or mRNA of tissues or cells from different sources, and reverse transcript them into double stranded cDNA, and then use restriction endonuclease to cut the cDNA from each source into DNA fragments of different lengths; ligate the cDNA enzymatic products from each source with DNA adapters that can differentiate the sources, making the cDNA of each source labeled with DNA adapters of different sequences. DNA adapters are composed of two single strand DNA that are not completely complementary to each other, and its structure is shown in FIG. 2. That's to say, one of its ends contains sequence 1 that is complementary to the cut of the above restriction endonuclease; and it ligate with double stranded cDNA enzymatic fragments in the action of ligases; the other end is “Y” structure, which is made up of a pair base sequences 2 and 3 that are not complementary to each other. Sequence 3 is also can be designed to the one complementary to sequence 2, but in this case, the 3′ terminus of sequence 3 must be properly modified to make it won't perform extension reaction in the action of polymerase. Sequence 2 contains a gene source-specific sequence 4, and sequence 5 that won't change with gene sources is between this sequence and the 5′ terminus of this strand. Different gene source-specific DNA adapters can be designed into such state that the base sequence is only different at sequence 4, but the type and number of bases forming this sequence are the same.
The second method is reverse transcription primer labeling method. That's to say, first extract the total RNA or mRNA of tissues or cells from different sources, and reverse transcript them into cDNA with primers of different sequences, making cDNA from each source labeled with DNA fragments of different sequences. The structure of reverse transcription primer is shown in FIG. 3. Its 3′ terminus (sequence 1 in the Figure) is composed of multiple thymines, and a gene source-specific sequence 2 is between the 3′ terminus and the 5′ terminus, and a base sequence 3 that does not change with gene sources is between this sequence 2 and the 5′ terminus of this strand. Different gene source-specific reverse transcription primers can be designed into such state that the base sequence is only different at sequence 2, but the type and number of bases forming this sequence are the same.

(2) PCR Amplify the Same Gene from Different Sources on an Equal Proportion Basis.
Usually, the expression level of the target gene extracted from tissues is small and it can be detected only by PCR amplification. One of the key technologies of this patent is how to amplify the above labeled gene from different sources in a monotube on an equal proportion basis. First we should design a gene specific primer (GSP) according to the sequence of the target gene; meanwhile design another common primer (CP), and its sequence is the same as the 5 sequence (when labeled with DNA adapters) in FIG. 2 or the sequence 3 (when labeled with revere transcription primer) in FIG. 3. In the condition that primers CP and GSP are present, if some source contains the target gene then GSP will first anneal with it and extension reaction occurs, the primer CP anneals with the extension product and extension occurs; if there is no extension product of GSP, then primer CP won't extend. A pair of primers CP and GSP is used to amplify the same gene fragment from different sources and the Tₘ values of the amplified products are totally the same (the length and the base species are the same), so the PCR amplification can be ensured on an equal proportion basis.
If the arm 2 and arm 3 in FIG. 2 are complementary to each other, then the arm 3 will extend in the action of DNA polymerase, producing the template for CP annealing. Therefore, the PCR amplification of the same gene from different sources cannot be realized on an equal proportion basis.

(3) The Sequencing of the Amplified Products of the Same Gene from Different Sources.
At present the commonly used sequencing reaction is based on the principle of gel electrophoresis, which is qualitative detection, and the quantitative characteristic is bad; furthermore, it cannot detect the base sequence following the primer, that's to say, it cannot detect the sequences of the first 50 bases. Bioluminescent assay is based on PPi detection. For example, pyrosequencing is a method for sequencing by orderly adding each dNTP in cycle, which can not only directly detect the base sequence following the primer, the quantitative characteristic is also very good, and the number of the repetitive bases can be determined by measuring the peak height. The present invention detects the sequences of the amplified products of the same gene from different sources by sequencing method based on PPi. The base type in sequence can be used to differentiate the gene source, and the peak intensity can be used to judge the gene expression level difference of different sources. Gene expression level analysis is the important content of genomics research. The purpose of the present invention is to apply the sequencing technology to the comparative assay of gene expression level difference. Compared with the present technology, the innovation of the invention is: the expression level difference of the same gene from different individuals can be detected by only one assay, and additional detection cost is not required. Easy to be instrumented, laser, gel, fluorescence labeling, and electrophoresis are not required. This inventive method has a wide application prospect for its advantages of high sensitivity, good quantification, low price and simple operation. This method has a significant meaning for the screening of disease-related genes, clinical early diagnosis and the preparation of specific medicine for the treatment of disease. And it also can be used to study the expression of the relative genes when human beings, animals or cells are in the treatment of drugs or other methods.

### BRIEF DESCRIPTION OF THE DRAWINGS

FIG. 1 is the principle chart of detecting the difference of gene expression level between various sources of the present invention.

FIG. 2 is the structural schematic diagram of DNA adapters.

FIG. 3 is the structural schematic diagram of reverse transcription primer.

FIG. 4 is the sequencing result when using DNA adapters to label the P53 genes in human brain cancer tissue, normal tissues and liver cancer tissue.

FIG. 5 is the sequencing result when using reverse transcription primers to label the P53 genes in human liver cancer cell and human bladder cancer cell.

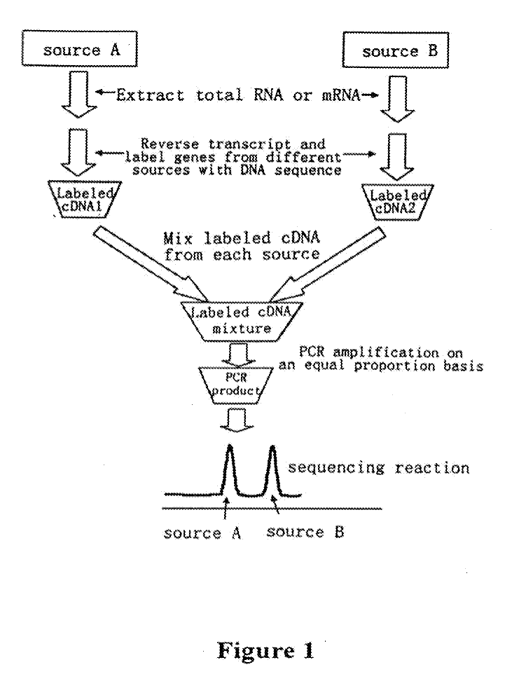

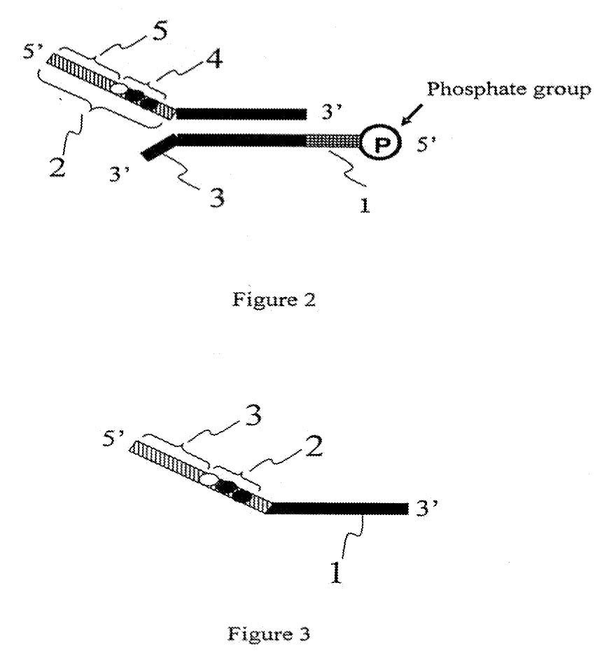

### DETAIL DESCRIPTION OF THE INVENTION

Concrete examples are used to illustrate the above method, the main experimental steps are as below:

(1) Label the same gene from different sources. Respectively extract the total RNA or mRNA of relative tissues or cells from each individual and detect its concentration. Then according to DNA adapter labeling method or reverse transcription primer labeling method, make the cDNA from each source labeled with DNA adapters of different sequences or DNA fragments of different sequences. When labeling with DNA adapter labeling method, first reverse transcript the mRNA into double stranded cDNA; cut the cDNA into fragments of a certain length by restriction endonuclease (Mob I) that can identify the sequences of the four bases. Respectively ligate these fragments with the DNA adapters containing gene source-specific sequences. Then mix cDNA fragments of different sources that are labeled with DNA adapters, and use the mixture as the template of PCR amplification reaction. When labeling with reverse transcription primer labeling method, respectively reverse transcript mRNA into cDNA with the reverse transcription primers that are corresponding to each source, mix after purification, and use the mixture as the template of PCR amplification reaction.

(2) PCR amplification. Perform PCR amplification reaction on the DNA template in (1) using a common primer (CP) that is not relevant to gene source and a gene specific primer (GSP). Because a pair of primers CP and GSP are used to amplify the same gene from multiple sources, the relative proportion of the given gene in the each source is fixed in the amplification process, that's to say, the amplification is performed on an equal proportion bases, and the proportion won't change with the increase of amplification times. If the expression level difference of multiple genes from the above different sources needs detecting, different GSP needs to be added to perform the amplification.

(3) Quantitatively detect the amplification products of the gene fragments from different sources at the same time. After the single strand is prepared from the amplified PCR product by biomicrosphere technique or restriction enzyme digestion, add the common primer complementary to the template sequence to anneal. Or directly purify the PCR products, and then add the common primer complementary to the template sequence to anneal. Then detect the sequences of two bases by bioluminescence method, namely respectively add dNTP or ddNTP corresponding to the gene sources to the solution containing substrate. If the added dNTP or ddNTP is complementary to the template, then PPi will be released. PPi is converted into ATP rapidly in the action of enzyme, and ATP reacts with luciferin in the action of luciferase to produce light signal. In the result, the base sequence represents the different gene source, and the signal intensity represents the gene expression level of each source. According to the gene expression level difference of each individual, we can quickly judge the function of genes and find the disease-related functional genes.

### EMBODIMENT 1

The detection of gene expression level difference of P53 gene in human normal tissue, brain cancer tissue and liver cancer tissue.

This example detects the expression levels of P53 genes of the tissues from three different sources by DNA adapter labeling method. First, design three different DNA adapters, respectively ligate them with the cDNA fragments digested by restriction endonuclease, and then mix to perform PCR amplification.

1. The Preparation of cDNA Sample.

(1) The extraction of total RNA: respectively get 0.1 g human normal tissue, brain cancer tissue and liver cancer tissue, add 1 ml Trizol to the Tissuelyser to grind, extract total RNA according to the operation in the Trizol instruction. Identify it by electrophoresis, if the 28 s and the 18 s bands are complete and have no degradation, detect its concentration by ultraviolet absorption method, and then regulate its final concentration to 1 μg/μl with sterile DEPC-H₂O.

(2) The synthesis of the first strand cDNA: Oligo(dt)₁₆ (100 μmol/L) 1 μl and total RNA (1 μg/μl) 3 μl, the mixture is incubated at 70° C. for 10 min, place it on ice, add 4 μl the first strand buffer solution of 5 times' concentration, 2 μl DTT (0.1 mol/L), 1 μl Rnase inhibitor (40 U/μl), 4 μl dNTP mixture (2.5 mmol/L each), 4 μl DEPC-H₂O, incubate at 37° C. for 2 min, add 1 μl Superscript II (200 U/μl), incubate at 42° C. for 1 h, 70° C. for 10 min, cool it down on ice.

(3) The synthesis of the double stranded cDNA: add 30 μl the second strand buffer solution of 5 times' concentration, 12 μl dNTP mixture (2.5 mmol/L each), 1 μl E coli ligase (10 U/μl), 10 μl DNA polymerase I (4 U/μl), 1 μl RNase H (2 U/μl) to the above mix solution, add DEPC-H₂O until the total volume reaches 150 μl, incubate at 16° C. for 2 h, 70° C. for 10 min.

2. Label the Same Gene from Different Sources.

(1) Enzyme digestion reaction: add 10 μl double stranded cDNA, 2 μl buffer solution of 10 times' concentration, 1 μl Mbo I TaKaRa endonuclease (10 U/μl), 7 μl distilled water for sterilization, the total volume of the reaction system is 20 μl. Place the mixture in 37° C. water bath and react for 2 h, and then place it at 70° C. for 10 min to inactivate the Mbo I enzyme. The feature of the Mbo I endonuclease is that it can identify the 5′→3′ GATC order in DNA, and cut it to form the GATC adhesive end with the 5′ terminus bumps.

(2) Ligation reaction: get equal volume of endonuclease reaction solutions of different sources; respectively ligate them with 3 different DNA adapters. One strand adp-4 of the three DNA adapters is the same, another strand contains four gene source-specific bases, and the four bases only have different sequence, all of them are composed of c, t, g, and c. Their sequences are: adp-1: 5′-ccc cac ttc ttg ttc tct cat gtca cg cat cac tcg-3′, adp-2: 5′-ccc cac ttc ttg ttc tct cat ctga cg cat cac tcg-3′; adp-3: 5′-ccc cac ttc ttg ttc tct cat atcg cg cat cac tcg-3′; adp-4: 5′-gat ccg agt gat gcg cta ag-3′. The parts having underlines and italic are gene source-specific bases. adp-1 and adp-4 form a DNA adapter 1, adp-2 and adp-4 form a DNA adapter 2, adp-3 and adp-4 form a DNA adapter 3. All of the adapters have the structure with the 5′ terminus bumps the four bases GATC. DNA adapters 1, 2 and 3 are respectively used to label the P53 genes in human normal tissue, brain cancer tissue and liver cancer tissue. Get 1 μl enzyme digestion solution, add 2 μl two single strands (10 p mol/L) that form the adapters (2 μl each), 2 μl 10×T4 DNA ligase Buffer and 11 μl distilled water for sterilization, place the mixture at 70° C. for 10 min, and then cool down the temperature to 16° C. at the rate of 0.2° C./s, add 2 μl T4 DNA ligase (4 U/μl) and react for 2 h.

3. PCR Amplification and the Preparation of the Single Strand.

(1) PCR amplification: mix the above ligation products from three different sources into one reaction tube at the raton of 1:1:1, and respectively add 2 μl common primer (CP, 5′-ccc cac ttc ttg ttc tct cat-3′) (10 pmol/L), 2 μl specific primer (5′-gga gca cta agc gag cac tg-3′) (10 pmol/L) of P53 gene labeled by biotin, 3 μl Mg²⁺ (25 mmol/L), 4 μl dNTP Mixture (2.5 mmol/L each), 5 μl 10× PCR Buffer and 0.5 μl TaKaRa Taq DNA polymerase, and then add distilled water for sterilization until the total volume reached 50 μl to perform PCR amplification. The conditions for the PCR reaction are: 94° C. 30 s, 60° C. 30 s, 72° C. 30 s, the reaction is carried out for 35 cycles. The finally obtained product is the double stranded DNA labeled by biotin.

(2) The preparation of single strand: get 25 μl M280 beads and wash them according to the requirements of operation instruction. Dissolve in 50 μl 2× B&W Buffer (wash buffer), add equal volume of PCR product to react 30 min, shake slightly in the reaction process so as to make the beads in a suspension state. Fasten the beads with magnet and desert the supernatant, add 20 μl NaOH solution (0.1 mol/L) after washing the beads 2-3 times with 1× B&W Buffer, and then react for 5 min. Extract the supernatant to another tube and regulate the pH value to 6˜7 with diluted hydrochloric acid, and then store it in refrigeration. The solid phase beads are dissolved in the 1× B&W Buffer to store after being washed and leave it to use when sequencing.

4. Compare the Relative Expression Level of the Same Gene from Different Sources by Base Sequence Determination Method.

Prepare the solutions that contain 25 mM Mg²⁺ and 5 mM Tris(pH 7.7) from the above single strand DNA sample (the biomicrosphere in step 3), and respectively add 5 pmol CP into each solution, heat the solutions at 70° C. for 10 min and then naturally cool them down to room temperature. Get 1˜5 μl solution and add it into sequencing detection standard mixed solution of 100 μl, and then orderly add dNTP to perform sequencing reaction.

The composition of the sequencing detection standard mixed solution is: 0.1 M Tris-HAc (pH 7.7), 2 mM EDTA, 10 mM Mg (Ac)₂, 0.1% albumin (BSA), 1 mM dithiothreitol (DTT), 3 μM adenosine 5′ phosphosulfate (APS), 0.4 mg/ml polyvinylpyrrolidone (PVP), 0.4 mM fluorescein, 200 mU/ml adenosine triphosphate sulfurylase (ATP-sulfurylase), 2 U/ml apyrase, 1 U DNA polymerase Klenow without exonuclease activity.

5. Detection Results.

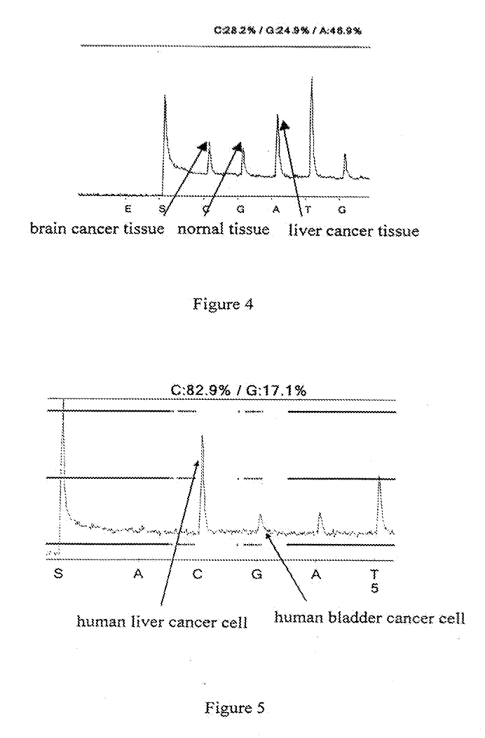

For DNA adapters 1, 2 and 3 are respectively used to label the P53 genes in human normal tissue, brain cancer tissue and liver cancer tissue, when adding dGTP, the obtained signal intensity represents the gene expression level from human normal tissue; when adding dCTP, the obtained signal intensity represents the gene expression level from human brain cancer tissue; when adding dATPaS (the analogue of dATP), the obtained signal intensity represents the gene expression level from human liver cancer tissue. The sequencing result is shown in FIG. 4. The first base “C” of the sequence in the Figure is from the DNA adapter 2, representing the expression level A1 of the P53 gene in human brain cancer tissue; the second base “G” is from DNA adapter 1, representing the expression level A2 of the P53 gene in human normal tissue; the third base “A” is from DNA adapter 3, representing the expression level A3 of the P53 gene in human liver cancer tissue. The ratio of peak heights of the three base sequences represents the expression level difference of the P53 gene in the three sources. The two times' detection results (A1:A2:A3) are: 28.20:24.9:46.9 and 28.1:22.4:49.5, average ratio (A1:A2:A3) is: 28.15:23.65:48.2.

### Embodiment 2

The detection of gene expression level difference of P53 gene in human liver cancer cell and bladder cancer cell.

This embodiment mainly use reverse transcription primer labeling method to detect the expression level difference of P53 gene in human liver cancer cell and bladder cancer cell. That's to say, use primers of different sequences to respectively reverse transcript mRNA from different sources, making the cDNA from different sources labeled with DNA fragments of different sequences. And compare the result with the RT-PCR detection result.

1. The Preparation of the Sample to be Detected.

Respectively extract total RNA from human liver cancer cell and bladder cancer cell according to the method in [Embodiment 1]. Identify it with electrophoresis, if the mass is complete, then detect its concentration by ultraviolet absorption method, and then regulate its final concentration to 1 μg/μl with DEPC-H₂O. Respectively use reverse transcription primers P-1 and P-2 to reverse transcript the mRNA in human liver cancer cell and bladder cancer cell into cDNA. The sequences of reverse transcription primers P-1 and P-2 are: P-1: 5′-ccc cac ttc ttg ttc tct cat cag ttt ttt ttt ttt ttt-3′ P-2: 5′-ccc cac ttc ttg ttc tct cat gac ttt ttt ttt ttt ttt-3′.

The reaction steps are: get 3 μl primer P-1 or P-2 (10 pmol/L) and 3 μl total RNA (1 μg/μl), place at 70° C. for 10 min, then place it on ice, add 4 μl the first strand buffer solution of 5 times' concentration, 2 μl DTT (0.1 mol/L), 1 μl Rnase inhibitor (40 U/μl), 4 μl dNTP mixture (2.5 mmol/L each), 2 μl DEPC-H2O, incubate at 37° C. for 2 min, and then add 1 μl SuperScript™ II RNase H—reverse transcriptase, incubate at 42° C. for 1 h, 70° C. for 10 min, cool it down on ice. Mix at equal volume after purification, and use the mixture as the template of PCR reaction.

2. PCR Amplification and the Preparation of Single Strand.

Perform PCR amplification and prepare single strand according to the method in [Embodiment 1]. Wherein the CP and gene specific primer are the same as that of [Embodiment 1].

3. Comparatively Assay the Gene Expression Level of the Same Gene from Different Sources by Base Sequence Determination Method.

Prepare the solutions that contain 25 mM Mg²⁺ and 5 mM Tris(pH 7.7) from the above single strand DNA sample, and respectively add 5 pamol CP into each solution, heat the solutions at 70° C. for 10 min and then naturally cool them down to room temperature. Get 1˜5 ml solution and add it into sequencing detection standard mixed solution of 100 ml, and then orderly add dNTP to perform sequencing reaction.

For reverse transcription primers P-1 and P-2 are respectively used to label the P53 genes in human liver cancer cell and bladder cancer cell, so when adding dCTP, the obtained signal intensity represents the gene expression level from human liver cancer tissue; when adding dGTP, the obtained signal intensity represents the gene expression level from human bladder cancer tissue.

4. Detection Result.

The sequencing result is shown in FIG. 5. The first base “C” of the sequence in the Figure is from the reverse transcription primer P-1, representing the gene expression level A1 of human liver cancer cell; the second base “G” is from the reverse transcription primer P-2, representing the gene expression level A2 of human bladder cancer cell. The ratio of peak heights of the two base sequences represents the expression level difference of the P53 gene in the two sources. The two times' detection results (A1: A2) are: 82.9:17.1, 87.4:12.6, 84.2:15.8, 89.5:10.5, average value is: 86:14=6.14:1, standard deviation of the detection is 3.0:3.0.

The expression levels of P53 gene in liver cancer cell and bladder cancer cell by RT-PCR method are 126359 copies/μl and 22093/μl, the ratio is 5.72:1. Compare the detection result of the two methods; the relative average deviation is less then 2%, which indicates that the detection result of the method in present invention is more accurate.

Indexing in article:
1. Bentley, D. R. 2000. The Human Genome Project—an overview. Medicinal Research Reviews 20: 189-96.
2. Zhang, L., W. Zhou, V. E. Velculescu, S. E. Kern, R. H. Hruban, S. R. Hamilton, B. Vogelstein, and K. W. Kinzler. 1997. Gene expression profiles in normal and cancer cells. Science 276: 1268-72.
3. Debouck, C., and P. N. Goodfellow. 1999. DNA microarrays in drug discovery and development. Nature Genetics 21: 48-50.
4. Velculescu, V. E., L. Zhang, B. Vogelstein, and K. W. Kinzler. 1995. Serial analysis of gene expression. Science 270: 484-7.
5. Karet, F. E., D. S. Charnock_Jones, M. L. Harrison_Woolrych, G. O_Reilly, A. P. Davenport, and S. K. Smith. 1994. Quantification of mRNA in human tissue using fluorescent nested reverse-transcriptase polymerase chain reaction. Analytical Biochemistry 220: 384-90.
6. Schena, M., D. Shalon, R. W. Davis, and P. O. Brown. 1995. Quantitative monitoring of gene expression patterns with a complementary DNA microarray. Science 270: 467-70.
7. Ronaghi, M., M. Uhlen, and P. Nyren, A sequencing method based on real-time pyrophosphate. Science, 1998. 281(5375): p. 363, 365.
8. Zhou Guohua, Gu Zhuoliang, Zhang Jiebin. Detect the mutation point on P53 gene by bioluminescent assay. Acta pharmaceutica Sinica, 2002; 37(1):41-45.
9. Guo-Hua Zhou, Masao Kamahori, Kazunori Okano, Kunio Harada, and Hideki Kambara. Miniaturized pyrosequencer for DNA analysis with capillaries to deliver deoxynucleotides. Electrophoresis, 2001, 22, 3497-3504.

What is claimed is:

1. A comparing method for expression amount of the same gene from different sources by base sequence measurement comprising:
(a) use DNA sequence labeling method to make the reverse transcript complementary DNA (cDNA) of mRNA from different sources contain a segment of source-specific sequence;
(b) mix the labeled cDNA of different sources into one tube and use it as the substrate of polymerase chain reaction (PCR); PCR amplification is performed using the same common primer and a gene-specific primer;
(c) Detect the base sequence of the above PCR amplified products corresponding to the gene sources by bioluminescent assay, wherein the base type represents the gene source, and the signal intensity of each base represents the gene expression level from each source.

2. The comparing method of claim 1, wherein the said different sources mean different tissues or cells.

3. The comparing method of claim 1, wherein the said DNA sequence labeling method means that restriction endonuclease is used to prepare double stranded cDNAs from different sources into fragments of proper length through enzymolysis; and then ligate the fragments with DNA adapters of different sequences—cDNA from different sources is ligated with different DNA adapters.

4. The comparing method of claim 3, said DNA adapter contains the sequence complementing to the cuts of the restriction endonuclease in claim 3, and is composed of two single strand DNAs that are not completely complementary to each other; and the adapter can ligate with double stranded cDNA enzymatic fragments in the action of ligases.

5. The comparing method of claim 4, wherein one of said two single strand DNAs that are not completely complementary to each other contains a segment of gene source-pecific sequence, and a base sequence that does not change with gene sources is between this sequence and the 5′ terminus of this strand, and this base sequence is not complementary to the 3′ terminus of another strand.

6. The comparing method of claim 1, wherein the said DNA sequence labeling method means that reverse transcript mRNAs from different sources with primers of different sequences respectively, making cDNAs from different sources labeled with different DNA fragments.

7. The comparing method of claim 6, wherein the said primers of different sequences means that the primer's 3′ terminus is composed of multiple thymines, and there is a gene source-specific sequence between the 3′ terminus and the 5′ terminus, and a base sequence that does not change with gene sources is between this sequence and the 5′ terminus of this strand.

8. The comparing method of claim 1, wherein the said common primer means that the primer's sequence is partly or completely similar with the base sequence (as mentioned in claim 5 and claim 7) that does not change with gene sources.

9. The comparing method of claim 1, wherein said bioluminescent assay means the method that quantitatively assay the pyrophosphate (ppi) produced by extension reaction.

10. The comparing method of claim 9, wherein said extension reaction means: use the PCR amplified product of claim [1] or its single strand product as template, add the sequencing primers to anneal, then orderly add dNTP, or ddNTP or their analogues, in the action of DNA polymerase, when the added dNTP or ddNTP or their analogues complementary to the template, the polymerization occurs.
